(19) United States
(12) Patent Application Publication
(10) Pub. No.: US 2001/0021394 A1
(43) Pub. Date: Sep. 13, 2001

(54) Induction of a cellular stress response with heavy metal salts

(76) Inventor: George A. Perdrizet, South Glastonbury, CT (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/765,731

(22) Filed: Jan. 11, 2001

Related U.S. Application Data
(63) Continuation of application No. 09/288,767, filed on Apr. 7, 1999, now abandoned.
Application No. 09/288,767 is a continuation of application No. PCT/US97/18088, filed on Oct. 7, 1997.
Application No. PCT/US97/18088 is a continuation-in-part of application No. 08/727,114, filed on Oct. 8, 1996, now Pat. No. 5,955,111.

(51) Int. Cl. A01N 59/16; A61K 33/24

(52) U.S. Cl. 424/617; 424/643; 424/650; 424/620; 424/630; 424/639; 424/644; 424/646; 424/652; 424/653; 424/654; 424/702

(57) ABSTRACT

The present invention relates to compositions and methods of induction of a cellular stress response and a protected phenotype using heavy metal ions. The invention relates to methods of protecting a mammal against injury caused by a noxious condition by administering to the mammal a heavy metal in sufficient quantity and under appropriate conditions to induce a sufficient cellular stress response to provide protection (partial or complete) against injury caused by a noxious condition. The invention also relates to methods of inducing a cellular stress response in a mammal by administering a heavy metal in sufficient quantity and under appropriate conditions to induce a cellular stress response to protect the mammal against injury caused by a noxious condition. The invention also relates to compositions comprising a heavy metal, and an additional component, such as a stress protein, agents which enhance a cellular stress response, aid in the uptake of heavy metal into the tissue, or have a synergistic effect with heavy metals, stress proteins and/or other agents for the induction of the cellular stress response.

### RELATED APPLICATIONS

This application is a continuation of PCT Application No. PCT/US97/18088, filed Oct. 7, 1997, which is a continuation-in-part of U.S. application Ser. No. 08/27,114, entitled “Compositions for Inducing Production of Stress Proteins by George A. Perdrizet, filed Oct. 8, 1996, the teachings of which are incorporated herein in their entirety by reference.

### BACKGROUND OF THE INVENTION

Individuals are affected by a wide variety of injuries and traumas, such as ischemia and reperfusion. It would be helpful to have additional methods or techniques for aiding individuals in their response to such events.

### SUMMARY OF THE INVENTION

The present invention relates to methods of using heavy metal ions to induce a stress response and a protected phenotype, which is a stress-induced cellular stress response providing a transient state of protection against injury caused by a noxious condition, as well as to compositions useful in the methods. The invention relates to methods of protecting a mammal, such as a human, against injury caused by a noxious condition by administering to the mammal a heavy metal in sufficient quantity and under appropriate conditions to induce a cellular stress response which protects (partially or completely) against injury caused by a noxious condition. The heavy metal is administered to the mammal in sufficient quantities to provide protection (partial or complete) against injury caused by the noxious condition. This transient state of protection against the effects of a noxious condition is known as the protected phenotype.

Noxious conditions are those which cause injury, including oxidant injury or damage to one or more organs, tissues or cells in animals, including humans, nonhuman animals, plants, foods and any other biological material. Such noxious conditions include, but are not limited to, ischemia, reperfilsion, hyperthermia, hypothermia, toxemia, oxygen and nutrient deprivation, heavy metal toxicity, ethanol toxicity, physical injury, hyperglycemia, radiation, oxygen toxicity, acute inflammatory states, anoxia, hypoxia, reoxygenation, drug toxicities, reactive oxygen species (ROS) including superoxide radicals, transportation-associated stress to livestock and severe physical or psychological stress. One form of transportation-associated stress is shipping fever, a respiratory disease complex of ruminants, also known as porcine stress syndrome.

The heavy metal is administered sufficiently prior to the injury to allow time for the induction of a cellular stress response sufficient to result in protection against the noxious condition. The heavy metal can also be administered during the injury to enhance the development of the protected phenotype. Heavy metals which are relatively nontoxic to humans, such as tin and zinc, are administered in particular embodiments. In one particular embodiment, stannous chloride is administered. Particular routes of administration include subcutaneous injection and intraperitoneal injection.

The invention also relates to compositions comprising a heavy metal and at least one other component, such as an agent which enhances a cellular stress response, aids in the uptake of heavy metal into the tissue or which has an additive or enhancing effect with heavy metals, stress proteins, or another agents to produce a protected phenotype.

### DETAILED DESCRIPTION OF THE INVENTION

Change in gene expression induced by cellular stress is known as the cellular stress response, or heat shock response (HSR). The response is a phenomenon in which adaptive, reversible changes in cellular metabolism are rapidly induced after exposure to a sublethal cellular stress. The development of the response is associated with a temporary period of protection, during which the cell, tissue, or organ is protected from what would otherwise be irreversible, or even lethal, injury. This transient state of protection is known as the protected phenotype. The purposeful induction of the cellular stress response to protect living tissues from the injurious effects of stress is known as stress conditioning.

Several features of the HSR indicate the utility of the present invention. First, the HSR is a universally prevalent, fundamental cellular response occurring in all species studied, from bacteria to humans. Therefore, it is likely that any cell type can be protected against injury or damage caused by noxious conditions by appropriate manipulation of this response. Second, the HSR protects the cell, tissue, and whole organism against the injurious effects of a subsequent lethal exposure to the stressful agent; that is, a state of acquired tolerance to stress (the protected phenotype) has been induced. Third, cross protection (also known as cross-resistance or cross-tolerance) exists. That is, exposure to a sublethal, preconditioning stress, for example, hyperthernia, can confer protection against other forms of seemingly unrelated stress, such as cold ischemia, hypoxia, tumor necrosis factor, and various cellular toxins. This means that even if the exact nature of the harmful condition or noxious agent is not understood, it is still possible to provide protection against it. Fourth, an optimal period can be determined from the time of exposure to the sublethal conditioning stress to the time of exposure to the harmful condition or noxious agent, thereby allowing sufficient time for the maximal development and expression of the protective response.

This invention relates to inducing the protected phenotype in a mammal by administering to the mammal a heavy metal in sufficient quantity and under appropriate conditions to induce a cellular stress response in the mammal, in order to protect against injury and to aid in recovery. The stress response induced is sufficient to produce the desired effect of protection against a noxious condition, which can occur simultaneously with, or after administration of the heavy metal. The invention also relates to protecting a mammal against injury caused by a noxious condition by administering to the mammal a heavy metal in sufficient quantity and under appropriate conditions for induction of a cellular stress response which protects against injury caused by the noxious condition. This cellular stress response can aid in recovery of normal cellular function.

Induction and enhancement of expression of stress proteins plays a role in the protective effect of the cellular stress response. This includes expression of inducible stress proteins and enhanced expression of baseline levels of constitutively expressed stress proteins. As used herein, a “stress protein”, also known as a “heat shock protein” (HSP), is a protein encoded by a stress gene (heat shock gene); it is typically produced in significantly greater amounts upon contact or exposure to a source of stress. A “stress gene”, or “heat shock gene”, is used herein to mean a gene that is activated or which undergoes detectable increased expression by contact or exposure to stress or other effective stimuli.

One such stress protein is heme oxygenase (HO), or heat shock protein 32kd (HSP 32). Heme oxygenase is an enzyme which participates in degradation of heme to biliverdin, which then can be broken down to bilirubin, a highly effective antioxidant. There are two heme oxygenase isoenzymes, which are the products of two distinct genes. Heme oxygenase-l (HO-1) is the inducible form. Heme oxygenase-2 (HO-2) is a constitutively expressed form which is not induced by HO-1 inducers. HO-1 induction results in a decrease in microsomal heme and consequent modulation of important cellular functions. A binding site for the transcription factor NF-κβ, which activates aspects of the inflammatory response, including activation of heme oxygenase, is located in the human HO-1 promoter region. Induction of HO-1 during oxidative stress may restore the antioxidant/prooxidant ratio inside the cell and aid the cell in responding to oxidant injury. Other examples of heat shock proteins include HSP 110, HSP 104, HSP 90, HSP 70, HSP 65, HSP 56 and HSP 27. “Protection” can be partial or complete, and, as a result, the effects of the injury are less than they would be if the stress protein production had not been induced or enhanced. This invention will permit cells, tissues, organs, and organisms to survive what would otherwise be damaging or lethal events.

A “heavy metal” is defined herein as mercury, cadmium, tin, lead, zinc, arsenic, copper, nickel, selenium, iron, cobalt, manganese, vanadium, gold, gadolinium, indium, bismuth, titanium, or molybdenum. Preferred heavy metals are tin and zinc.

The heavy metal can be administered in any of the chemical forms in which it is available. The name of a specific heavy metal as used herein encompasses all forms of that metal. For example, the term “tin” includes a compound containing tin in the stannous oxidation state (Sn⁺²) or the stannic oxidation state (Sn⁺⁴). The heavy metal can be administered in combination with any anion, particularly a physiologically acceptable ion, including chloride, sulfide, citrate, acetate, oxide, carbonate, hydroxide, phosphate, sulfate and halides, e.g. chloride, bromide, fluoride, and iodide. Compounds can further be defined to include additional solvent molecules or neutral Lewis bases. The anions can be chelating or multidentate, such as oxalate (C₂O₄²⁻). A variety of tin compounds include stannous oxide, stannous acetate, stannous bromide, stannous chloride, stannous fluoride, stannous pyrophosphate, stannic oxide and stannic acetate. In specific embodiments stannous fluoride, stannous chloride and stannous pyrophosphate are administered. Suitable zinc compounds include zinc oxide, zinc chloride, zinc bromide, zinc acetate, zinc carbonate, zinc sulfate, zinc phosphate, zinc oleate, zinc stearate, zinc gluconate, and zinc valerate. In specific embodiments, zinc compounds are zinc oxide, zinc chloride, zinc acetate, zinc carbonate, zinc oleate, zinc stearate, zinc gluconate, and zinc valerate. The heavy metal can be administered in a composition. In one embodiment, stannous chloride is administered in a composition containing sodium chloride.

Factors to be considered in determining how a heavy metal will be administered for induction of cellular stress response include the time of the anticipated noxious event, the dosage of heavy metal and any accompanying agent, and route of administration. Appropriate conditions are those, including timing of administration of the heavy metal, which provide the desired effect protection against the injury from the noxious condition).

A heavy metal can be administered sufficiently prior to injury to allow for the induction of a sufficient stress response to provide protection against that injury, but should not be administered so far in advance of the injury that the degree of protection is inadequate to provide the prophylactic or therapeutic effect desired. This time frame is generally from one hour to one week, depending on the organism, the noxious condition, the heavy metal, and the conditions of administration. In another embodiment, a heavy metal may be administered during a noxious event.

The dosage of heavy metal or any accompanying agent administered will vary depending upon factors such as the phannacodynamic characteristics of the heavy metal and any accompanying agent(s), and its mode and route of administration; the age, health, and weight of the recipient; the nature and extent of symptoms, kind of concurrent treatment, frequency of treatment, and the effect desired. One dosage (SnC₂.H₂O, molecular weight 224.7) is approximately 0.15 mg/kg. of the recipient. In one embodiment, one dose is administered prior to surgery. An appropriate dosage for humans is in the range of 0.15 mg/kg to 112.5 mg/kg (0.00066 millimoles/kilogram to 0.5 millimoles/kilogram). In additional embodiments, more than one dose of heavy metal or a combination of heavy metals is administered as appropriate.

The particular physiological carrier in which the heavy metal and any accompanying agent are held in solution or suspension includes, but is not limited to, water, buffered saline, polyols (e.g., glycerol, propylene glycol, liquid polyethylene glycol) and dextrose solutions. The optimum concentration of the active ingredient(s), e.g., a heavy metal, in the chosen medium can be determined empirically, according to procedures well known to those of skill in the art, and will depend on the ultimate pharmaceutical formulation desired. Methods of introduction of heavy metals at the site of treatment include, but are not limited to, parenteral routes such as intradermal, intramuscular, intraperitoneal, intravenous, subcutaneous, intrathecal, intramedullary, and epidural; and nonparenteral routes such as transdermal, ocular, intranasal, oral, and rectal. Other suitable methods include biodegradable devices and slow release polymeric devices.

For parenteral administration, heavy metals can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles can also be used. The vehicle or lyophilized powder can contain additives that maintain isotonicity (e.g., sodium chloride, mannitol) and chemical stability (e.g., buffers and preservatives). The formulation is sterilized by known techniques. Suitable pharmaceutical carriers are described in the most recent edition of Remington's Pharmaceutical Sciences, A. Osol, a standard reference text in this field of art.

The heavy metal can be administered alone or in compositions which comprise, in addition to the heavy metal, at least one additional agent, such as, optionally, one or more agents which enhance or prolong the cellular stress response, one or more agents which aid in the uptake of the heavy metal into the desired tissue and one or more agents which have a synergistic relationship with heavy metals, stress proteins or other agents to produce heat shock gene expression. Agents which have a synergistic relationship with stress proteins are those that increase the expression or activity of the stress proteins. These compositions are also the subject of the present application. Such agents include, but are not limited to, chelating agents, antioxidants, antiproliferative prostaglandin, mild hyperthermia, oxidizing agents, allopurinol, vitamins, corticosteroids, surfactants, nonsteroidal anti-inflammatory drugs such as aspirin, amino acids such as glutamine and glycine, and anabolic hormones such as growth hormone and insulin growth factor 1.

Ischemia is anoxia or local anemia due to obstruction of blood flow. The loss of blood flow through tissues results in the buildup of toxic metabolic waste products and is associated with the loss of intracellular high energy compounds and acidosis. Ischemia causes a marked decrease in the intracellular content of high energy phosphates. The loss of cellular energy stores results in mitochondrial dysfunction and a reduction of Na⁺/K⁺-ATPase pump activity which in turn leads to a disruption of the normal electrochemical gradients across cellular membranes. The net result is an influx of Na⁺ ions and water into the cell and cellular swelling. Loss of ionic gradients disrupts membrane transport functions and signaling. Cellular membranes become abnormally permeable and a rapid influx of Ca²⁺ions occurs, resulting in uncontrolled enzymatic activation and other damage. Cell death occurs by both necrotic and apoptotic pathways. Ischemia can increase oxidant injury as well, by decreasing the levels of native free radical scavengers, such as superoxide dismutase, glutathione and catalase, and by increasing production of toxic reactive oxygen intermediates, also known as reactive oxygen species or superoxide radicals. Ischemia can produce or initiate an inflammatory response.

Reperfusion is the return or repassage of blood or other fluid through a vascular bed. Reperfusion injury is fundamentally an inflammatory lesion and, therefore, may represent a nonspecific inflammatory response which occurs as ischemic tissues become reoxygenated. This is due largely to the endogenous production of toxic reactive oxygen intermediates, which provide direct and indirect damage to cellular membranes, enzymes, nonenzymatic proteins, and nuclear contents. They can cause lipid peroxidation and initiate a chain reaction of auto-oxidation events leading to membrane damage. This permits excessive calcium influx into the cell, uncontrolled enzymatic activation, and eventual cell death.

The common thread linking a number of these injury syndromes, such as noncardiogenic pulmonary edema following cardiopulmonary bypass, hemorrhagic shock and trauma, organ preservation and acute allograft rejection, may be oxidant injury. The human body has intrinsic antioxidant protection, including the mitochondrial-cytochrome oxidase system, enzymatic forms including superoxide dismutase (SOD), catalase, glutathione peroxidase, and heme oxygenase, and nonenzyme forms.

One purpose of the HSR may be to protect the host tissues against inflammatory consequences of ischemia/reperfusion. HSR is associated with preservation of glutathione reductase levels and induction of superoxide dismutase activity, both of which could conceivably provide protection to the ischemic organ subjected to reperfusion. Stress proteins avidly bind nucleosides and nucleotides including adenosine triphosphate (ATP) and are involved in transportation of proteins across mitochondrial and other cellular and subcellular membranes. Also, stress proteins function in unstressed cells as molecular chaperones which guide and assist in the proper folding and translocation of complex protein molecules within and between cellular compartments. The HSR may involve the peptide binding functions of stress proteins which may prevent intracellular protein denaturation and allow transmembrane transportation of macromolecules during times of stress and permit rapid restoration of enzymatic function, during a noxious insult or once the noxious condition has been removed.

Ischemia and reperfusion are caused by a number of events, including many modern invasive surgical and medical techniques which result in the interruption of blood flow to tissues and organs. For example, the methods of the present invention would be useful for treatment before surgical procedures such as cardiopulmonary bypass, coronary artery bypass, peripheral vascular bypass, complex aortic surgery, carotid artery surgery, renal artery surgery, coronary artery angioplasty and difficult obstetric procedures, for example as a means to prevent fetal and neonatal brain injury from hypoxia. In each of these situations, organs and tissues are consistently damaged through ischemia and reperfusion. These methods would also be useful for intervention in the care of an injured patient, especially a patient with head or spine injury, to prevent secondary injury. These methods would also be useful for treatment of patients with established coronary artery disease or cerebrovascular disease. The invention can also be used to protect against ischemic necrosis of pedicled skin flaps as used in plastic surgery and during limb amputations. A pedicled skin flap is a skin flap which is attached to a stalk through which the flap receives nourishment.

In addition, many diagnostic and therapeutic agents and procedures used in clinical medicine expose organs and tissues to toxic side effects. Cellular protection by the present invention can prevent organ and tissue damage from drug related and other toxicities. One such example is the use of intravenous radiocontrast materials currently used in x-ray imaging and chemotherapeutic agents. These compounds are known to be toxic to the kidney and frequently induce renal dysfunction in patients. It may be possible to protect kidneys from injury due to these compounds if the stress response is induced prior to patients receiving these toxic agents. In a similar fashion, undesirable toxic side effects of current chemo- and radiation therapies for cancer could be prevented or reduced by the present invention.

Induction of stress proteins by the present invention will protect tissues and organs from acute global inflammatory states. For example, it can protect isolated cells from cytotoxicity by mediators of sepsis such as interleukin-1, tumor necrosis factor, and bacterial endotoxins. Sepsis is the systemic inflammatory response to infection. There are a number of patients who are readily identifiable as being at high risk for developing sepsis and SIRS (Systemic Inflammatory Response Syndrome), such as those with multiple trauma, patients on mechanical ventilators, premature babies, immunosuppressed patients and patients undergoing complex gastrointestinal surgery.

Induction of a cellular stress response by heavy metals including stannous chloride can serve a number of other purposes as well, such as the enhanced preservation of fruits, fish and meats for storage and transportation, since many of these foods spoil due to oxidant injury. The present invention can also be used to prepare individuals for conditions of extreme physical activity such as those that are encountered during surgery, battle or war, sporting events or space flight. This would allow individuals to temporarily sustain damaging or otherwise lethal forces or injuries. It may also be useful to prepare people for physical or psychological stress, such as athletic competition, and the professional activities of government leaders, business executives, airline pilots, air traffic controllers, and high performance teams. It would also be useful for preventing or reducing the harmful effects of transportation-associated stress in animals.

In addition, the teachings of the present invention can be used to assess the ability of an animal to produce certain stress proteins, and for research about stress proteins, heat shock response and the protected phenotype.

The present invention will now be illustrated by the following examples, which are not intended to be limiting in any way.

### EXAMPLE I

### Acute Spinal Cord Ischemia in the Rabbit

Materials and Methods

New Zealand white rabbits were stress conditioned and subjected to spinal cord ischemia using methods similar to those previously described in Perdrizet et al., “Stress conditioning: A novel approach to organ preservation”, Current Surgery, 46(1): 23-26 (January-February 1989), the contents of which are incorporated herein by reference in their entirety. The rabbits were subjected to spinal cord ischemia by occlusion of the infrarenal-abdominal aorta as an ischemic insult to the spinal cord, for 20 minutes at 37° C. The occlusion was then removed and blood flow re-established to the infra-renal aorta. Neurological function of the lower extremities was evaluated 24 hours after reperfusion. Four animals were pretreated with a subcutaneous injection in the rump region of stannous chloride (SnCl₂) in a saline diluent at a dose of 0.15 mg per kilogram (0.00066 millimoles per kilogram), sixteen hours prior to aortic occlusion.

Results

Control animals were consistently paralyzed. Animals stress conditioned with whole body hyperthermia showed intact neurologic function in eight of eight animals tested. Seven animals were completely normal, and one had intact function but lacked normal strength. Two animals were kept for long-term follow up: One animal to three months and one animal to six months. At follow up dates, both animals were completely normal. Twenty four hours after reperfusion, all four animals pretreated with SnCl₂ had normal neurologic function.

### EXAMPLE II

### Warm Ischemic Injury of the Rat Kidney

Materials and Methods

Male Sprague-Dawley rats were stress conditioned and subjected to warm ischemic injury using methods similar to those previously described in Perdrizet et al., supra. The non-treated control animals underwent 60 minutes of occlusion of the renal artery at 35-37° C. as an ischemic insult. Following the ischemic injury, flow was reestablished in the kidney and vascular resistance of the kidney was measured. To measure renal vascular resistance, a syringe system was attached to the renal artery, to give a constant flow rate of approximately 0.5 cc/minute. Since there was a constant flow rate, it was possible to measure the pressure generated in the syringe. The higher the pressure, the greater the resistance. Renal vascular resistance was recorded as mm mercury/ml fluid/gram of tissue.

Results

Renal vascular resistance of the uninjured non-ischemic kidney averages approximately 43-44 resistance units. Following 60 minutes of warm renal ischemia, this resistance increased to approximately 60 resistance units in the control group. The kidneys that were previously stress conditioned through whole body hyperthermia prior to warm renal ischemia, demonstrated a remarkable preservation of renal vascular resistance of 37 resistance units. The kidneys that were stress conditioned with stannous chloride demonstrated a renal vascular resistance of 45 resistance units following 60 minutes of warm ischemic injury.

### EQUIVALENTS

Those skilled in the art will recognize, or be able to ascertain using no more than routine experimentation, many equivalents to the specific embodiments of the invention described specifically herein. Such equivalents are intended to be encompassed in the scope of the following claims.

I claim:

1. Use of a heavy metal for the manufacture of a medicament for use in:
a. stress conditioning;
b. protecting a mammal against injury caused by a noxious condition;
c. inducing a protected phonetype (e.g. in a mammal); and
d. inducing a cellular stress response sufficient to protect a mammal against injury from a noxious condition.

2. Use of claim 1 wherein the noxious condition is any of: ischemia, reperfusion, hyperthermia, hypothermia, toxemia, oxygen deprivation, nutrient deprivation, heavy metal toxicity, ethanol toxicity, traumatic injury, hyperglycemia, radiation, oxygen toxicity, acute inflammatory states, anoxia, hypoxia, drug toxicity, shipping fever, oxidant injury, oxidation injury, physical stress and psychological stress.

3. A composition comprising a heavy metal and at least one agent selected from the group consisting of:
a. agents which enhance a cellular stress response;
b. agents which enhance the protected phenotype;
c. agents which aid in the uptake of the heavy metal into a desired tissue; and
d. agents which have a synergistic effect with a heavy metal, a stress protein or an agent as defined in a, b or c.

4. The use of claim 1 or claim 2, or the composition of claim 3, wherein the heavy metal is selected from any of: mercury, cadmium, tin (e.g. in the stannous or stannic oxidation state), lead, zinc, arsenic, copper, nickel, selenium, iron, cobalt, manganese, vanadium, gold, gadolinium, indium, bismuth, titanium and molybdenum.

5. The use or composition of claim 4 wherein the tin is in the form of a compound selected from any of: stannous fluoride, stannous chloride and stannous pyrophosphate.

6. The use or composition of claim 4 wherein the zinc is in the form of a compound selected from any one of zinc oxide, zinc chloride, zinc acetate, zinc carbonate, zinc oleate, zinc stearate, zinc gluconate and zinc valerate.
